(19) United States
(12) Patent Application Publication
(10) Pub. No.: US 2019/0212335 A1
(43) Pub. Date: Jul. 11, 2019

(54) AIE Nanoparticle Conjugates And Methods Therefor

(71) Applicant: Luminicell Pte. Ltd., Singapore (SG)

(72) Inventors: Hadhi Wijaya, Singapore (SG); Guangxue Feng, Singapore (SG); Bin Liu, Singapore (SG); Ben Zhong Tang, Kowloon (HK)

(21) Appl. No.: 16/093,000

(22) PCT Filed: Apr. 14, 2017
(86) PCT No.: PCT/IB2017/000431

§ 371 Date: Oct. 11, 2018

Related U.S. Application Data
(60) Provisional application No. 62/323,594, filed on Apr. 15, 2016.

(51) Int. Cl. G01N 33/543; A61K 49/00

(52) U.S. Cl. CPC G01N 33/54346; A61K 49/0093; A61K 49/0058

(57) ABSTRACT

Described are compositions comprising amphiphilic polymer nanoparticles, such as DSPE-PEG, encapsulating a photostable agent with aggregation-induced emission (AIE) characteristic. The photostable AIE agents are preferably small organic molecules with tetraphenylethylene moieties. The nanoparticles are synthesized by a modified nanoprecipitation method and the size of the nanoparticles is controlled by varying the loading ratio, the solvent ratio and the tatio of hydrophilic to hydrophobic length of the polymer. The nanoparticles are surface modified with a conjugatable group for covalently linking to at least one targeting moiety, such as antibodies or affibodies to IgG, EGFR and Her2. Methods for immunostaining or imaging or detecting or tracking a live cell, such as cancer cells, using the nanoparticle compositions are described.

This application claims the benefit of U.S. Provisional Application No. 62/323,594, filed on Apr. 15, 2016. The entire teachings of the above application are incorporated herein by reference.

Organic nanoparticles fabricated from fluorogens with aggregation-induced emission characteristics (AIE fluorogens) have received broad attentions as a promising platform for fluorescence bioimaging. These AIE fluorogens are non-emissive in molecular dispersed state in good solvents, but can be induced to emit strong fluorescence in aggregated or dry state. This unique AIE feature makes it possible to fabricate ultrabright AIE fluorogens based organic nanoparticles (AIE NPs) with excellent water dispersiblity and good photostability for biological applications. These nanoparticles generally lack specificity for cells or any biological event because they do not have surface targeting groups.

On the other hand, the antibodies have been extensive used for targeting specific proteins for studying and understanding the functions of different proteins as well as the interactions between them. Fluorescence tagged antibodies have become powerful vehicles for these studies. Small organic dyes including Cy3, FITC, and Alexa etc. have dominated this field; however, they tend to be quickly bleached under laser excitation, largely limiting their performance for long term study. While, semiconducting nanocrystal quantum nanoparticles (QDs) possess high brightness and much improved photostability, their intrinsic toxicity originated from their integral components has been raised as a big concern. Thus, the novel fluorescent AIE NPs can serve as promising candidates for the development of next generation of immunostaining reagents by conjugation with antibodies on their surface.

The ability to tune absorption/emission wavelengths of AIE fluorogens not only allows them to be excited with compatible common lasers to achieve optimal emission, but also offers the opportunity for multiplexed detection, which further simplifies detection process and reduces instrumental cost. Tetraphenylethylene (TPE) based AIE emitters are of interest. These molecules could be synthesized in only few steps from commercially available materials with tunable absorption and emission wavelengths and high quantum yields of up to unity. The color of the structure represents the corresponding emission of the AIE fluorogens: blue, green and red, respectively. See FIG. 1. These molecules are synthesized and their structures are confirmed using nuclear magnetic resonance spectroscopy (NMR) and elemental analysis.

### BRIEF DESCRIPTION OF THE DRAWINGS

The foregoing will be apparent from the following more particular description of example embodiments, as illustrated in the accompanying drawings. The drawings are not necessarily to scale, emphasis instead being placed upon illustrating embodiments.

FIG. 1 shows molecular structures of AIE fluorogens with tunable optical features. The color of the structure represents the corresponding emission of the AIE fluorogens: blue, green and red, respectively.

FIG. 2 is an illustration of AIE NP formation. Here the medium size means that the size is larger than 25 nm and ultra-small size is less than 5 nm.

FIGS. 3A-3C are graphs of the optical properties of the nanoparticles. Normalized UV (solid) and photoluminescence (PL) (dashed) spectra of the synthesized NPs in water (FIG. 3A blue, excited at 357 nm; FIG. 3B green, excited at 423 nm; FIG. 3C red, excited at 506 nm).

FIG. 4 shows laser light scattering data of the synthesized nanoparticles.

FIG. 5 is a schematic illustration of protein/antibody conjugation to AIE NPs.

FIGS. 6A-6F show UV (solid) and PL (dashed) spectra (FIGS. 6A-6C) and size distribution (FIGS. 6D-6F) of blue (FIGS. 6A, 6D), green (FIGS. 6B, 6E) and red (FIGS. 6C, 6F) AIE-IgG nanoparticles, respectively.

FIG. 7A shows fluorescence quantum yield changes of the three AIE-IgG nanoparticles upon 18 days incubation at 4° C. FIGS. 7B-7D show size distributions of blue (FIG. 7B), green (FIG. 7C), and red (FIG. 7D) AIE-IgG before and after 18 days incubation at 4° C.

FIG. 8A shows UV-vis and PL spectra of red AIE-EGFR and AIE-Her2 nanoparticles. FIG. 8B shows fluorescence quantum yields changes of red AIE-EGFR and AIE-Her2 nanoparticles upon continuous incubation at 4° C. FIGS. 8C and 8D show size distribution of AIE-EGFR (FIG. 8C) and AIE-Her2 (FIG. 8D) nanoparticles before and after 18 days incubation at 4° C.

FIG. 9 shows fluorescence intensity changes of human IgG upon incubation with red AIE-IgG or QD655-IgG with varied concentrations.

FIG. 10 shows fluorescence intensity changes of human IgG upon incubation with green AIE-IgG with varied concentrations.

FIG. 11 shows confocal images of MDA-MB-231 breast cancer cells after treatment with green AIE-EGFR nanoparticles, red AIE-EGFR nanoparticles, or red AIE dot without EGFR antibody conjugation. The cells were treated with these nanoparticles at concentration of 2 nM for 2 h at 37° C.

FIG. 12 shows confocal images of SKBR-3 breast cancer cells and NIH-3T3 fibroblast normal cells after incubation with red AIE-Her2 conjugates for 2 h at concentration of 2 nM.

FIG. 13 shows tracing of living SKBR-3 cells using confocal imaging by AIE670-Her2 or QD655-Her2 after 4 h incubation at concentration of 2 nM, and then subcultured for designated generation.

FIG. 14 shows confocal images of SKBR-3 breast cancer cells and NIH-3T3 fibroblast normal cells after incubation with green AIE-Her2 conjugates for 2 h at concentration of 2 nM.

FIG. 15A shows TPA cross section of green AIE-EGFR nanoparticles. FIG. 15B shows two-photon fluorescence image of MDA-MB-231 cells after treatment with green AIE-EGFR nanoparticles. FIG. 15C shows TPA cross section of red AIE-EGFR nanoparticles. FIG. 15D shows two-photon fluorescence image of MDA-MB-231 cells after treatment with red AIE-EGFR nanoparticles. These cells were treated with AIE-EGFR nanoparticles at a concentration of 2 nM for 2 h at 37° C. The two-photon fluorescence image is acquired with excitation wavelength of 820 nm; the green signal is collected between 540 to 580 nm; red signal is collected between 650 to 680 nm.

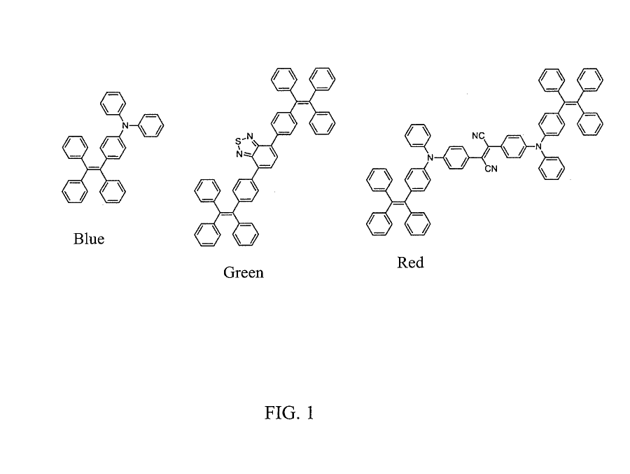

### DETAILED DESCRIPTION

In one embodiment is provided a nanoparticle composition comprising a plurality of surface conjugatable groups, wherein the nanoparticle comprises a biocompatible polymer shell having an average diameter of less than about 1000 nm, and a nanoparticle core encapsulated in the shell and comprising at least one uniform population of a photostable agent with aggregation-induced emission characteristic suitable for imaging applications; the polymeric surface of the shell comprising at least one conjugatable group; and optionally at least one targeting moiety that can specifically bind to a target, covalently linked to the at least one conjugatable group. In one aspect of the embodiment, the polymeric surface comprises at least one conjugatable group that is covalently linked to at least one targeting moiety that can specifically bind to a target. In another aspect of the embodiment, the polymeric surface comprises at least one conjugatable group that is not covalently linked to the at least one targeting moiety that can specifically bind to a target.

The biocompatible polymer shell can be any hydrophilic biocompatible polymer that can be surface modified with a conjugatable group. For examples, any of the FDA approved biocompatible hydrophilic polymers can be used, such as PEGₙ, where n is an integer between 10 and 1000, inclusive. Other biocompatible polymers are described in WO2013029340A9, for example at paragraphs [0130-0135], the entire teachings of this reference are incorporated herein by reference.

The core can comprise a hydrophobic lipid surfactant, such as 1,2-distearoyl-sn-glycero-3-phosphoethanolamine (DSPE). Examples of core materials are described in WO2013029340A9, for example at paragraphs [0130-0135], the entire teachings of this reference are incorporated herein by reference.

A portion of the surface can be functionalized with conjugatable groups. For example, at least about 10%, about 20%, about 30%, about 40%, about 50%, about 60%, about 70%, about 80%, about 90% of the surface is derivatized. The term “about” in this context means +/−0.5%.

In one embodiment, the nanoparticle has an average diameter of about 50 nm to about 300 nm, for example, about 50 nm. In another embodiment, the nanoparticle has an average diameter of about 20 nm to about 30 nm. In yet another embodiment, the nanoparticle has an average diameter of about 10 nm to about 20 nm. The term “about” as used in this context is intended to mean +/−5 nm.

The photostable agent with aggregation-induced emission characteristic has tunable absorption or emission wavelengths. In some embodiments the photostable agent with aggregation-induced emission characteristic has a chemical structure set forth in any one of the formulae I-III:

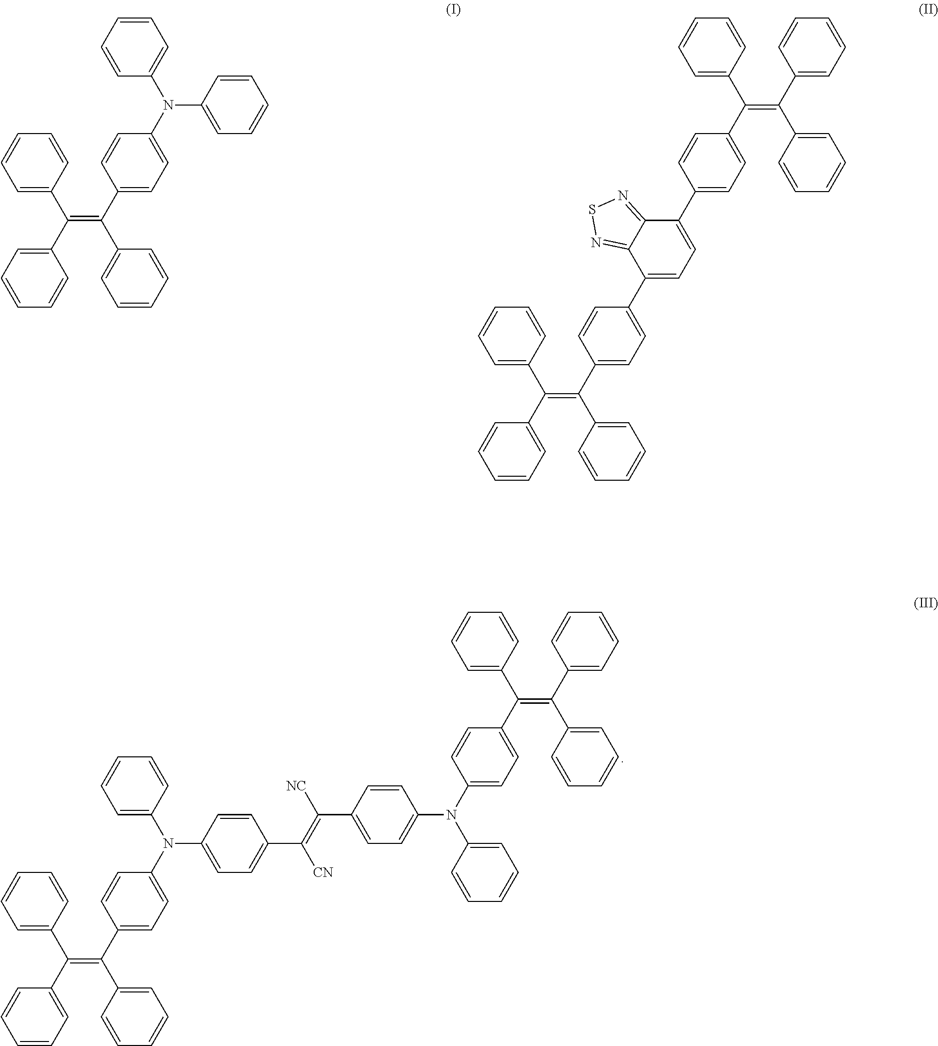 [SMILES: c1ccc(C(=C(c2ccccc2)c2ccc(N(c3ccccc3)c3ccccc3)cc2)c2ccccc2)cc1 ; c1ccc(C(=C(c2ccccc2)c2ccc(-c3ccc(-c4ccc(C(=C(c5ccccc5)c5ccccc5)c5ccccc5)cc4)c4nsnc34)cc2)c2ccccc2)cc1 ; N#C/C(=C(/C#N)c1ccc(N(c2ccccc2)c2ccc(C(=C(c3ccccc3)c3ccccc3)c3ccccc3)cc2)cc1)c1ccc(N(c2ccccc2)c2ccc(C(=C(c3ccccc3)c3ccccc3)c3ccccc3)cc2)cc1]

One of more of the hydrogen atoms on the one or more of the tetraphenylethylene moieties can be substituted with an electron group, such as methoxy, or electron withdrawing group, such as nitro or cyano.

The at least one conjugatable group can be, but is not limited to, an amine group, a carboxylic acid group, a sulfhydryl group, a maleimide group, an oxime group, alkyne, azide or combinations thereof. Other functional groups can be used provided that they can be conjugated to a targeting moiety.

The covalent linkage can be, but is not limited to, a peptide linkage, an amide linkage, a sulfhydryl linkage, a maleimide linkage, a thioester linkage, an ether linkage, an ester linkage, a hydrazine linkage, a hydrazine linkage, an oxime linkage or combinations thereof.

The targeting moiety can be, but is not limited to, a ligand, a biomolecule, protein, a specific recognition element, such as a peptide, aptamer, antibody, antigen or antigen binding fragment thereof, such as an affibody. The targeting moiety can be selected to recognize a specific marker or receptor on the target, for example, on the cell membrane. In one embodiment, the antigen binding fragment is an affibody, such as an anti-her2 affibody. In another embodiment, the antibody is an anti-EGFR antibody that binds to the epidermal growth factor receptor.

The target can be, but is not limited to, a surface antigen, ligand or receptor of a live cell, such as a cancer cell.

In another embodiment is provided a method for immunostaining or imaging a live cell, the method comprises a) contacting a live cell with a nanoparticle-target moiety complex, wherein the nanoparticle-target moiety complex comprises: a nanoparticle as described herein covalently linked to a targeting moiety; b) stabilizing the nanoparticle-target moiety complex that is bound to the live cell; c) exciting the photostable agent in the nanoparticle-target moiety complex that is bound to the live cell with a laser source capable of producing light with a specific wavelength and collecting the images; and d) processing the images, thereby imaging a live cell.

In one embodiment of the method, the targeting moiety can be, but is not limited to, a ligand, biomolecule, protein, a specific recognition element, such as a peptide, aptamer, antibody, antigen or antigen binding fragment thereof. In one embodiment, the antigen binding fragment is an affibody, such as an anti-her2 affibody. In another embodiment, the antibody is an anti-EGFR antibody that binds to the epidermal growth factor receptor.

The target can be, but is not limited to, a surface antigen, ligand or receptor of a live cell, such as a cancer cell.

In another embodiment is provided a method for controlling the size of a nanoparticle, the method comprises a) varying the loading ratio of the polymer to the dyes with aggregation induced emission; b) changing the solvent ratio (e.g., tetrahydrofuran to water ratio) used for the formulation of the nanoparticles; and c) changing the ratio of the hydrophilic to hydrophobic length of the polymer, to thereby control the size of a nanoparticle.

In another embodiment is provided a method for fine-tuning the nanoparticle size, color and surface functionality depending upon the desired properties and intended use of the nanoparticles, such as for immunostaining, cell specific cancer detection, multiphoton imaging, cell tracking, for example, cancer cell tracking. The color of the nanoparticle will depend upon the AIA fluorogen incorporated into the nanoparticle. The surface functionality will depend on the terminal group of the polymer used for the encapsulation. In an embodiment is provided a method for designing an AIE nanoparticle, comprising: selecting an AIE fluorogen that fluoresces at a desired wavelength; selecting a conjugatable group and linker that can be covalently linked to at least one targeting moiety; and controlling the size of the nanoparticle using the methods described herein.

In yet another embodiment is provided a kit for AIE nanoparticle conjugation to a targeting moiety, the kit comprises: a) surface functionalized AIE nanoparticle as described herein wherein the polymeric surface comprising at least one conjugatable group that is not covalently linked to the at one least targeting moiety; b) conjugation buffer; c) washing buffer; and d) instructions for performing the conjugation reaction, such as, for example, the conjugation protocols described herein.

The nanoparticle conjugates can be used in immunostaining, cell specific cancer detection, multiphoton imaging, cell tracking, for example, cancer cell tracking. In some embodiments, the targeting moiety is attached to the surface functionalized nanoparticle. In other embodiments, the surface functionalized nanoparticle is capable of but not yet conjugated to the targeting moiety. In this embodiment configuration, the researcher, investigator or the like can attach a targeting moiety of their own choosing, using, for example, the methods, kits and nanoparticles described herein.

### Fabrication of AIE Nanoparticles

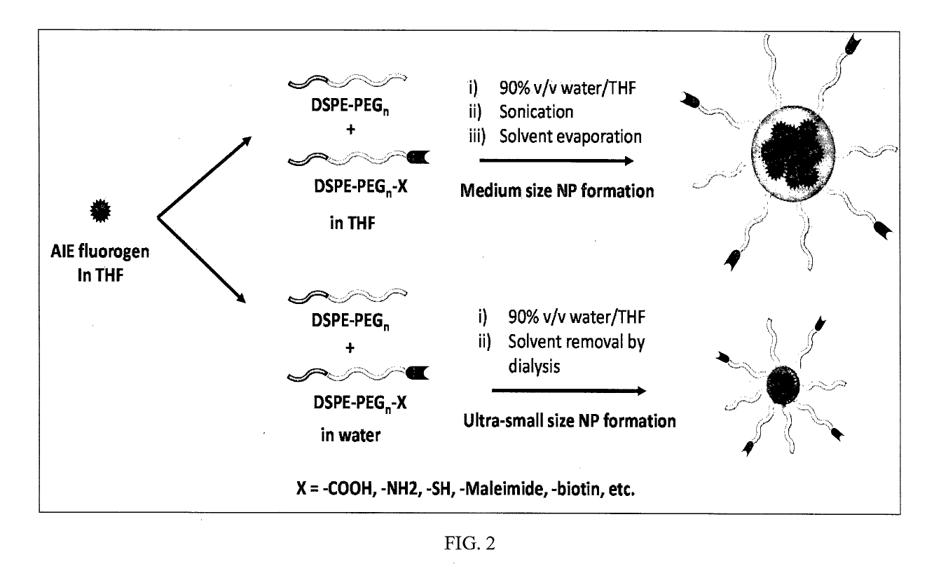

AIE nanoparticles with amendable surface functional groups were fabricated through polymer encapsulation strategy by using a modified nano-precipitation method (FIG. 2). 1,2-distearoyl-sn-glycero-3-phosphoethanolamine—Polyethylene glycol (DSPE-PEG) and its derivatives with different terminal functional groups (e.g., —COOH, —NH₂, —SH, -maleimide, -biotin, alkyne, azide, oxime, etc., and combinations of these) terminated at PEG chain will be used as the encapsulation matrix. The length of PEG can vary, for example, about 10 to about 1000 PEG units. Although DSPE-PEG is illustrated, the method is not limited to DSPE-PEG. Any amphiphilic block copolymer can also be used in the methods of making the nanoparticles. To form AIE nanoparticles, briefly, AIE fluorogens (such as, for example, the fluorogens of Formulae I-III), DSPE-PEG and its derivative will be dissolved in a homogeneous solution in THF solvent. This mixture will be added into MilliQ water at THF/Water ratio of 1/9, under ultrasound sonication. Upon mixing and ultrasonication, the hydrophobic DSPE segments will intertwine with AIE fluorogens to form the core, while PEG chains will extend outside towards the water phase to form the shell. These functional groups terminated at PEG ends will serve as the surface functional groups, ready for further conjugation.

Specifically, to synthesize ultra-small AIE NPs with size around 10 nm, 1 mL dilute THF solution containing the AIE fluorogens (0.1 mg/mL) is added into 10 mL aqueous solution containing the encapsulation matrix DSPE-PEGₙ-X and DSPE-PEGₙ where n is an integer between 10 and 1000, inclusive (1 mg/mL). The term “ultra-small” is intended to mean an AIE NP having an average diameter of about 10 nm to about 20 nm. The mixture is further sonicated in water bath sonicator to produce a homogeneous solution. The DSPE-PEG derivatives will serve as the surfactant and matrix to encapsulate AIE fluorogen aggregates to form the ultra-small AIE NPs. The mixture is further dialyzed against water to remove THF and excess DSPE-PEG derivatives. The suspension will then be centrifuged to remove the precipitated large aggregates. The suspended solution with sub-10 nm fluorescent NPs will be collected for characterization.

For the synthesis of medium NPs with size around 30 nm, AIE fluorogens and DSPE-PEG derivatives are molecularly dissolved in THF solution at the mass concentration of 1 mg/mL for AIE fluorogens and 2 mg/mL for DSPE-PEG derivatives, respectively. Then, 1 mL THF mixture of AIE fluorogens and DSPE-PEG derivatives was then added into 9 mL of aqueous solution under ultrasound sonication. The ultrasound sonication is prolonged for 2 min to promote the mixing and AIE NP formation. During the mixing and the ultrasound sonication, the hydrophobic DSPE segments will intertwine with AIE fluorogens to form the core, while PEG chains will extend outside towards the water phase to form the shell. The term “medium” is intended to mean an AIE NP having an average diameter of about 20 nm to about 50 nm.

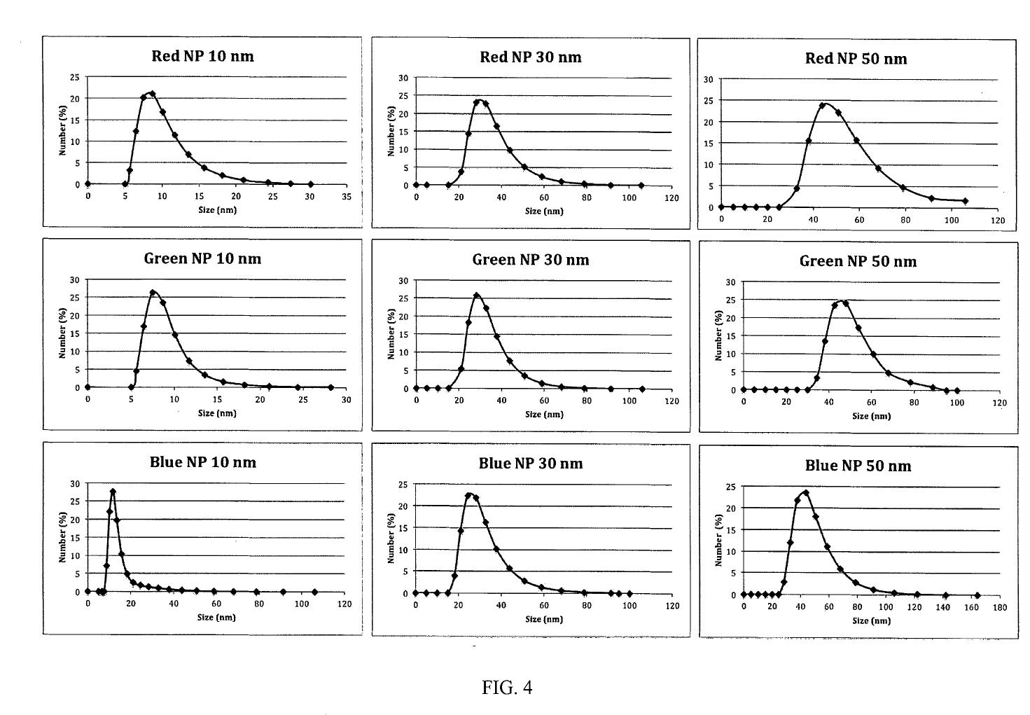

The large AIE NPs with size around 50 nm is synthesized following the same experimental procedures, but increasing the AIE fluorogen concentration in THF solvent to 1.35 mg/mL while keeping all other conditions unchanged. Laser light scattering (LLS), is used to study the NP size and size distribution, as shown in FIG. 4, the AIE NPs with desirable controlled sizes are successfully achieved. The term “large” is intended to mean an AIE NP having an average diameter of about 50 nm to about 300 nm.

### Optical Properties of the Nanoparticles

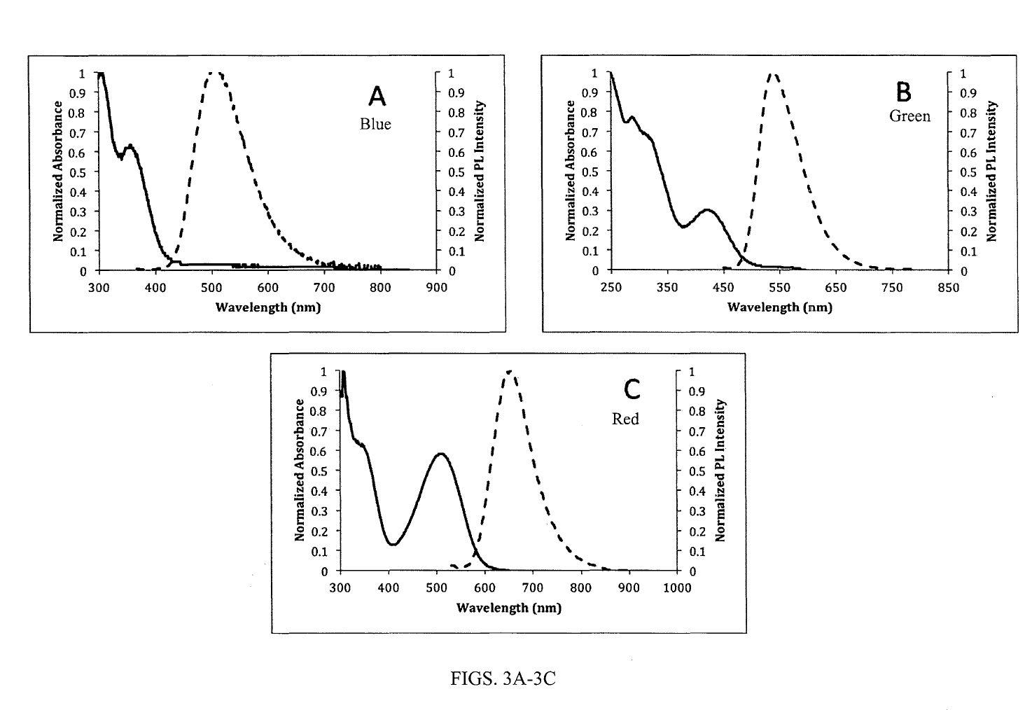

Blue, green and red NPs with tunable sizes have been successfully fabricated. For each color, the absorption and emission maxima of these NPs are not dependent on size. The absorption maxima of these fluorogens are between 350 and 550 nm (FIG. 3). The UV and PL spectra of these NPs indicate that they have large Stokes shifts and are therefore useful for cell imaging applications. The excitation wavelengths of these NPs are also compatible with currently available imaging system. Different polymers can be utilized as the encapsulation matrix to provide nanoparticles (NPs) with various surface functional groups (e.g., NH₂/COOH/maleimide). FIG. 4 shows the light scattering result for the representative nanoparticles with different colors.

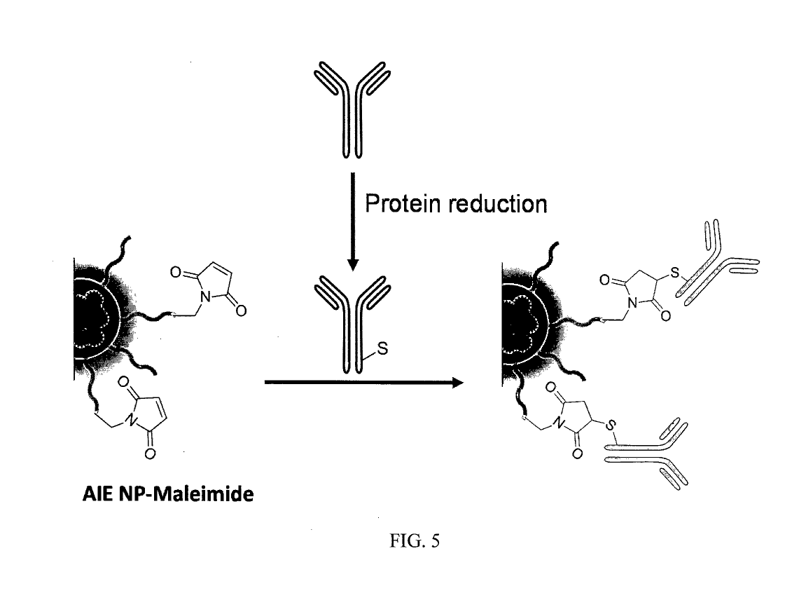

The synthesized AIE nanoparticles with terminal functionalities can be easily modified with various ligands and biomolecules for in vitro and in vivo imaging and diagnostic applications. One of the most common approaches is to utilize the general coupling reaction between the carboxyl-functionalized AIE nanoparticles and amine-bearing protein using activated reaction with N-ethyl-N′-dimethylaminopropyl-carbodiimide (EDC). However, this conjugation method may cause crosslinking between proteins due to the presence of large number of free carboxyl and amine groups. To suppress the undesired side reactions and eliminate multiple protection and de-protection steps, we chose an alternative approach to utilize the highly reactive and selective click reaction between thiol and maleimide groups (FIG. 5). The maleimide group can be easily introduced to AIE dot surface by changing the terminal group located at PEG chain end. While the thiol groups can be introduced to the protein via reduction reaction such as fragmentation by dithiothreitol (DTT) to expose free sulfhydryls or through a linker Traut's reagent (2-iminothiolane) to convert amine group to thiol group. Otherwise, commercially available thiol-modified ligand or protein can be used directly. Conjugates are concentrated by ultrafiltration and purified by size exclusion chromatography.

Here, we conjugated Goat Anti-Human IgG to the AIE nanoparticle surface as an example to demonstrate the conjugation procedures. Six μL of Traut's reagent (1 mg/mL) (purchased from Sigma Aldrich) was reacted with 150 μL of IgG antibody (1 mg/mL) (Thermo Fisher Scientific Inc., MA, USA) to introduce thiol group to antibody. After 1 h reaction, the mixture is centrifuged at 7500 rpm for 10 min using a filter tube with molecular cutoff of 10 kDa to remove the excess of the Traut's regents. The supernatant is discarded, and the precipitated antibody is washed with 0.4 mL of 1× PBS and centrifuged again at 7500 rpm for 10 min. The purified IgG antibody is dissolved in 0.5 mL of 1× PBS and further reacted with AIE nanoparticles (0.02 nmol) for 2 h at room temperature. The conjugation reaction is quenched by adding 10 μL of diluted 2-mercaptoethanol (add 3 μL of 2-mercaptoethanol to 4 ml of 1× PBS) to the solution and incubation for 30 min. Unreacted IgG antibody was removed by centrifuge at 7500 rpm for 10 min twice with filter tube with molecular cutoff of 300 kDa. The final conjugates are collected and diluted with 1× PBS to 0.5 mL. In addition, epidermal growth factor receptor (EGFR) antibody and thiol-modified Her2 affibody were also successfully introduced to AIE dot surface using the same strategy.

### Characterization of AIE Nanoparticles

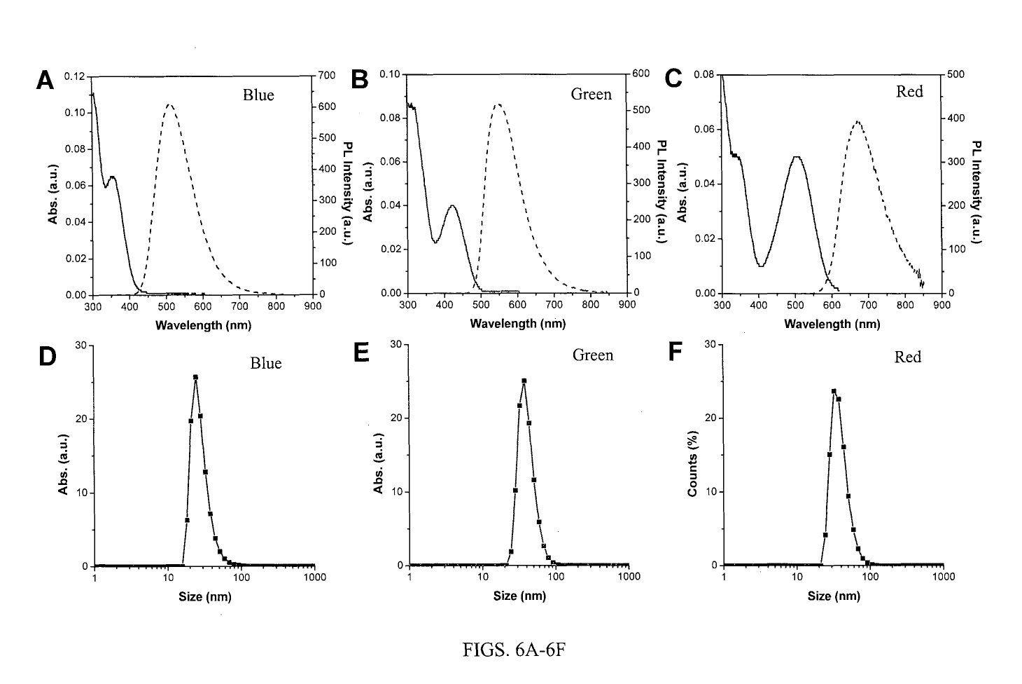

Three AIE-IgG conjugates with different colors are fabricated using the same protocol by simply changing the AIE fluorogens associated with different emissions. Their UV-vis absorption and emission spectra are shown in FIGS. 6A-6F. The absorption maximum is located at 356 nm, 422 nm and 510 nm, for blue, green, and red AIE-IgG conjugates, respectively. In addition, the green conjugate is excitable by commercial 405 nm, 457 nm, 488 nm lasers, while red conjugate is excitable by commercial 405 nm, 457 nm, 488 nm, 543 nm lasers. Such a broad choice of excitation lasers make them promising for varies confocal microscope. FIGS. 6A-6F also show the photoluminescence spectra, where the emission peaks are located at 510 nm, 540 nm and 670 nm, for blue, green and red AIE-IgG conjugates, respectively. Based on their emission, in addition, all of these three conjugates possess high fluorescence quantum yields, whereas 42.1% for blue, 60.5% for green, and 23.1% for red AIE-IgG nanoparticles, respectively, using 4-(dicyanomethylene)-2-methyl-6-(4-dimethylaminostyryl)-4H-pyran (QY=43%) as reference. The size of the three AIE-IgG nanoparticles was also studied, by dynamic light scattering. All of them have similar size distribution with an average diameter of ˜36 nm.

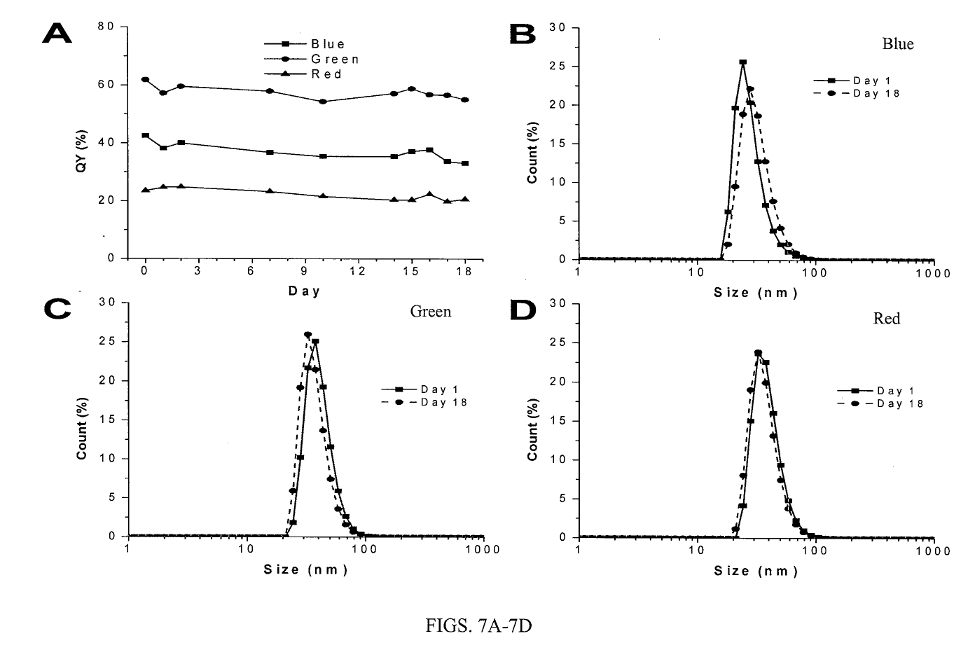

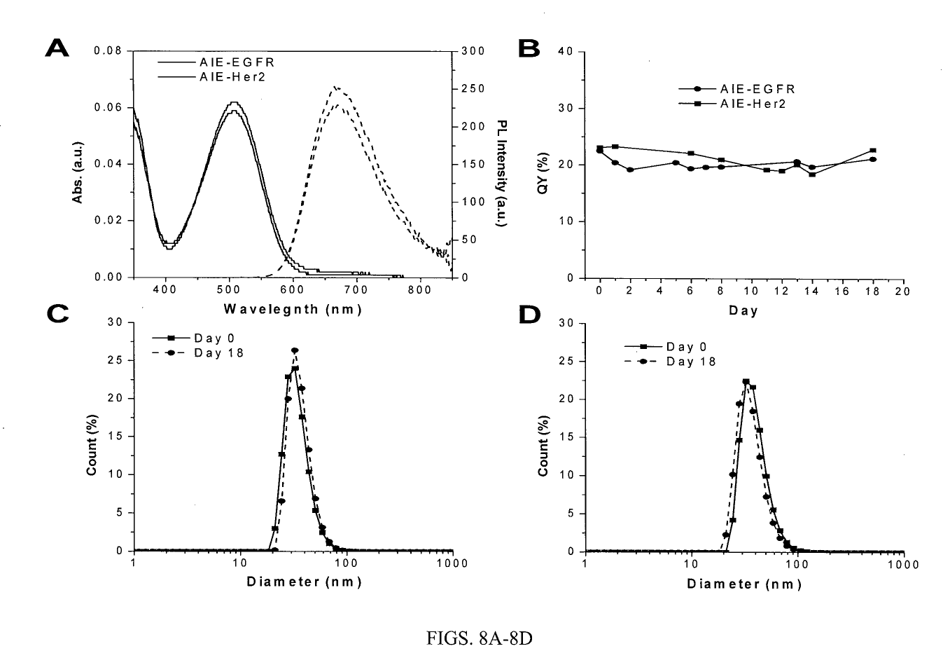

We further evaluated the stability of AIE-IgG nanoparticles. Their absorption and emission spectra (kept at 4° C.) were collected on daily intervals for up to 18 days, which is used for calculation of the fluorescence quantum yield changes along with the incubation. As shown in FIG. 7A, AIE-IgG nanoparticles showed excellent fluorescence stability, where the 18 day culture cause minimal effects to their quantum yield. In addition, the sizes of these nanoparticles before and after 18 days culture were also analyzed by DLS. See FIGS. 7B-7D. The results indicate that no significant/drastic changes in the size of these AIE-IgG nanoparticles. It should be noted that this strategy is applicable to EGFR monoclonal antibody and Her2 affibody, where similar fluorescence quantum yields and nanoparticle sizes are observed. Moreover, using red AIE dot example, these AIE-EGFR and AIE-Her2 nanoparticles exhibited similar sizes, fluorescence quantum yields, and excellent stability as compared to AIE-IgG nanoparticles (See FIGS. 8A-8D). This also illustrates the generality of our strategy, which can be used for fabrication of antibody conjugated AIE nanoparticles with tunable emissions and long term colloidal and bright stability.

### Immunoassay by AIE-IgG Nanoparticles

Immunolabeling of tissues is generally performed using secondary labelling process due to the high versatility and maximum immunoreactivity between the target and unlabeled primary antibody. Anti-IgG secondary antibody and its fluorescence conjugates have been widely used for specific labelling of primary IgG antibody. Here we test the binding ability of our goat anti-human IgG conjugated AIE-IgG nanoparticles towards human IgG using red AIE670-IgG dot as an example. The commercially available anti-human IgG conjugated quantum nanoparticles 655 (QD655-IgG) was selected as the benchmark.

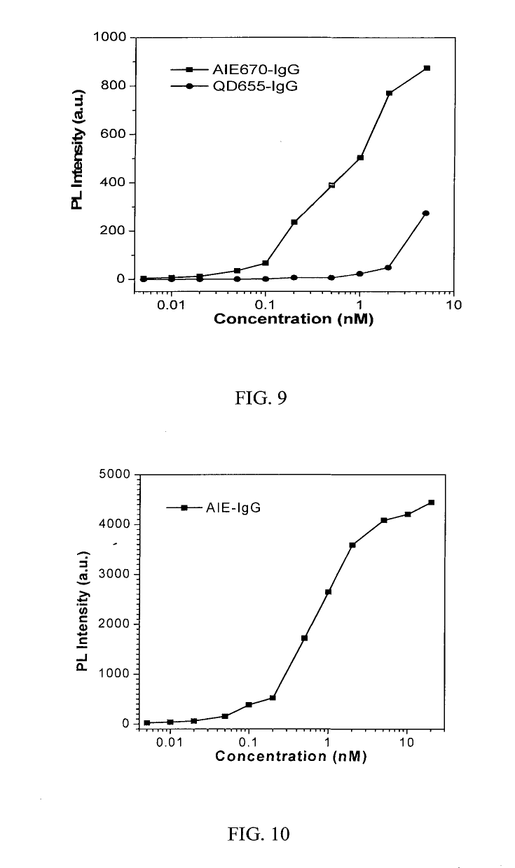

To perform the labelling, Human IgG was firstly seeded at the well bottom of the 96-well plate by incubation of 100 μL of Human IgG (1.2 μg/mL) per well at 4° C. After overnight incubation, the solution was discarded, and the well was washed twice with 0.05% Tween-20 in Tris-HCl buffer and blocked with 5% bovine serum albumin (150 μL) at 37° C. for 1 h. After washing, the red AIE-IgG nanoparticles or QD655-IgG was added into the 96-well plate (100 μL/well) with varied concentrations. After incubation at 37° C. for 30 min, the unbinding nanoparticles were removed, and the wells were washed three times, and the fluorescence intensity of 96-well plate is recorded by Microplate reader upon excited at 510 nm. As shown in FIG. 9, the fluorescence intensity of IgG significantly increases with the increase in AIE-IgG concentration, indicating the successful binding of AIE670-IgG towards human IgG. The commercially available QD655-IgG was also utilized as a control; however, the change in QD655-IgG fluorescence intensity is quite small when its concentration is below 5 nM. As a consequence, AIE-IgG nanoparticles show higher sensitivity in detecting IgG at the concentration ranging from 0.1 to 5 nM, compared with QD655-IgG. In addition, the green AIE540-IgG nanoparticles also show similar high sensitivity for IgG detection (FIG. 10).

Cancer Cell Imaging with AIE-EGFR Nanoparticles.

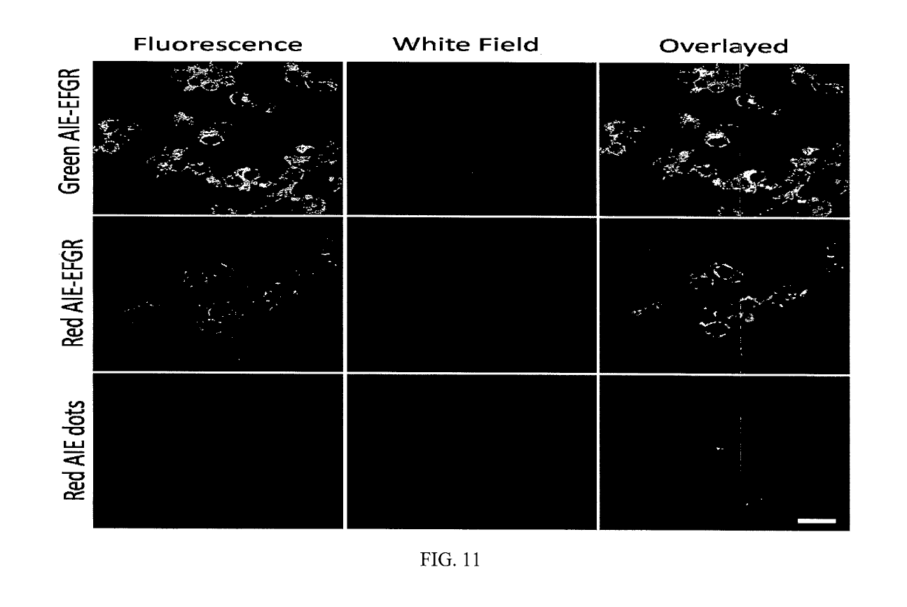

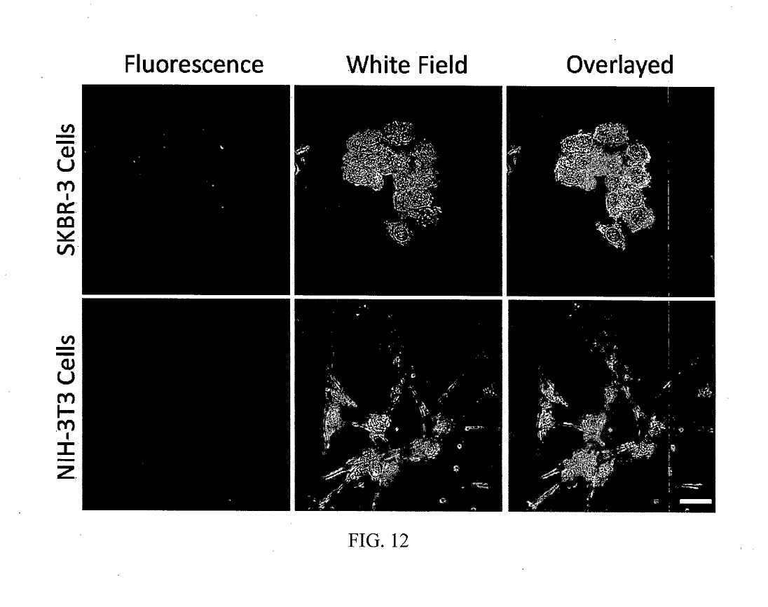

The epidermal growth factor receptor (EGFR) is a receptor tyrosine kinase of the ErbB family that is abnormally activated in many epithelial tumors. Fluorescence tagged EGFR antibodies are widely used for the detection of EGFR as wells for targeting cancer cell imaging with EGFR overexpression, but it was limited to small organic dyes based EGFR conjugates, whose fluorescence can be easily bleached by laser during the process of imaging. Our AIE nanoparticles have high brightness and excellent photostability, making them the ideal candidates for EFGR detection. Here we use our EGFR antibody conjugated AIE nanoparticles (AIE-EGFR nanoparticle) for detection and imaging of cancer cells with EGFR receptor overexpression. MDA-MB-231 breast cancer cells were selected as the demonstrating cell lines. The MDA-MB-231 cells were treated with green or red AIE-EGFR nanoparticles for 2 h at 37° C. We also treated the MDA-MB-231 cells with pure red AIE nanoparticles without EGFR antibody conjugation as control. FIG. 11 shows the corresponding confocal images. As indicated by the bright green and red fluorescence signals from MDA-MB-231 cells, the AIE-EGFR nanoparticles are able to successfully internalize into cells with EGFR overexpression. As control, the AIE nanoparticles without out EGFR decoration showed poor cellular uptake, where very weak red fluorescence can be observed inside cells. The results clearly demonstrated that the cellular uptake is mediated by the recognition of and binding to EGFR of the AIE-EGFR nanoparticles, and that our AIE-EGFR nanoparticles can be used for detection and imaging of cells with EGFR overexpression.

Targeted Cell Imaging with AIE-Her2 Nanoparticles.

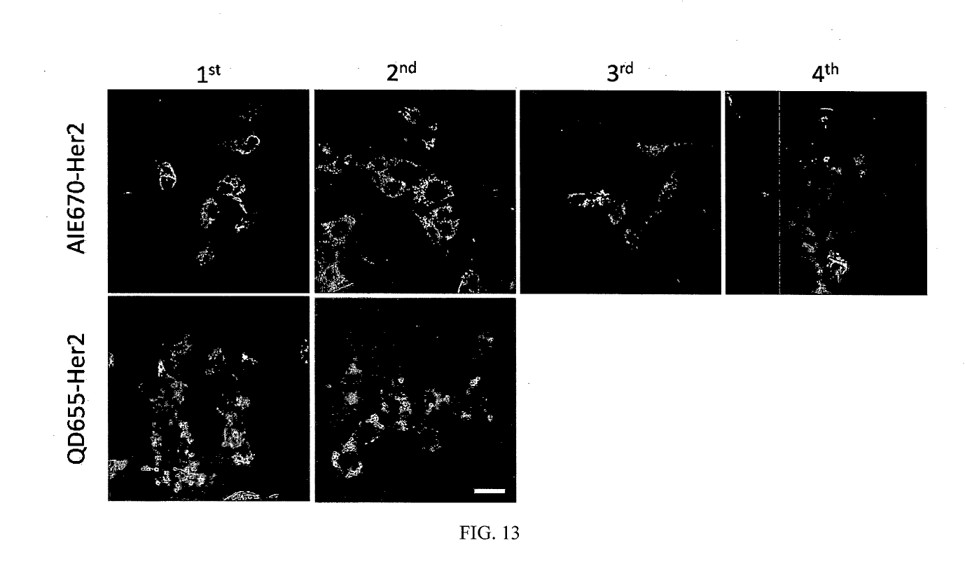

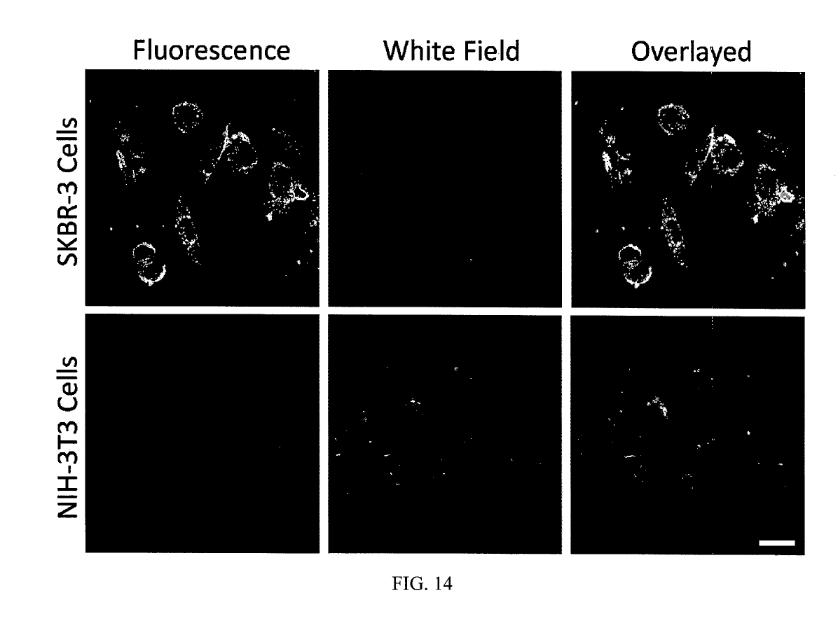

The human epidermal growth factor receptor HER2 (Her2/neu, ErbB2, or c-erb-b2) is a growth factor receptor that is expressed on many cell types. The Anti-HER2 Affibody® molecule is a highly specific affinity ligand selected against the extracellular domain of HER2. Here we demonstrated the excellent selectivity of Anti-Her2 affibody conjugated AIE nanoparticles (AIE-Her2 nanoparticles) towards Her2 overexpressed cancer cells (such as SKBR-3 breast cancer cells) over other cells lacking of Her2 expression (NIH-3T3 fibroblast cells were chosen as the negative control). Both cells are incubated with red AIE-Her2 nanoparticles (2 nM) at 37° C. for 2 h. After removing unbound AIE-Her2, the cells were imaged by laser scanning confocal microscope (LSCM, Olympus). As observed in FIG. 12, bright red fluorescence is observed in SKBR-3 cells, while negligible red fluorescence can be detected in control NIH-3T3 cells, clearly indicating the excellent selectivity of red AIE-Her2 towards cancer cells with Her2 overexpression. Quantitative analysis of the fluorescence intensity gives a 400% higher average brightness in AIE655-Her2 treated cells than those treated with QD655-Her2 conjugates. In addition, the fluorescence of QD655-Her2 is hardly observable in SKBR-3 cells at the 2nd generation, while AIE-Her2 brightness is tradable traceable up to 4 generations (More than twice longevity, FIG. 13). In addition, the green AIE-Her2 nanoparticles also showed the similar excellent selectivity towards SKBR-3 cancer cells (FIG. 14).

### Multiphoton Imaging

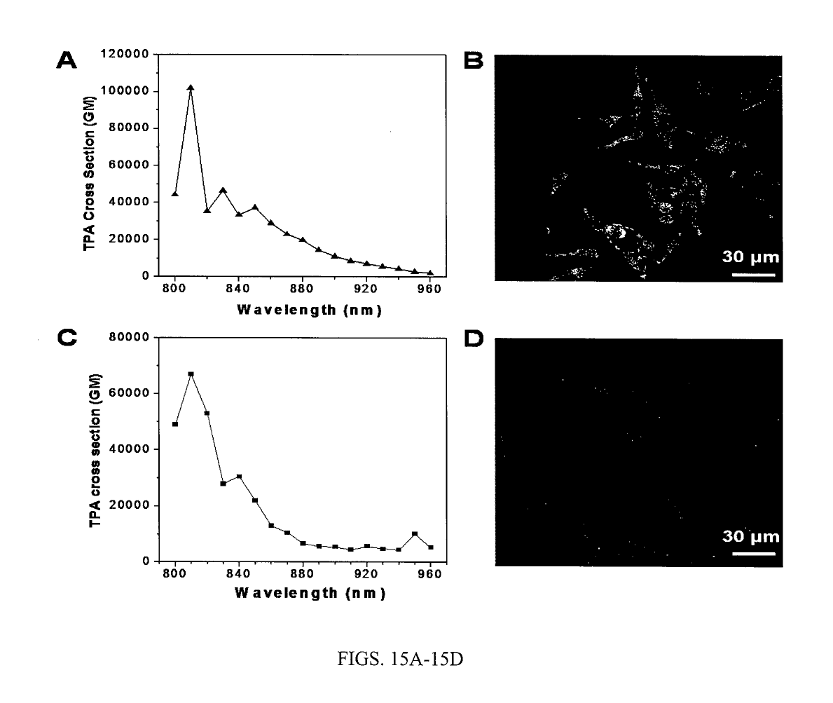

Fluorescent materials with a high two photon absorption (TPA) cross section could also be designed to emit strong visible fluorescence from low-energy irradiation in the FR/NIR region. This aspect of the fluorophore is particularly important in multiphoton microscopy for obtaining high resolution images within deep biological tissues. Here we measured the TPA spectra of both green and red AIE nanoparticles in aqueous solution using a multiphoton microscope equipped with a tunable Ti:sapphire pulsed laser, using Rhodamine 6G in methanol as the standard. As shown in FIGS. 15A and 15C, both green and red AIE nanoparticles showed a very high value of TPA cross section, where the maximum values are 10.2×10⁴ GM and 6.7×10⁴ GM for green and red AIE nanoparticles respectively. To demonstrate the great potentials of AIE nanoparticles in two-photon fluorescence imaging, MDA-MB-231 cells after 2 h treatment with green or red AIE-EGFR nanoparticles (2 nM) were fixed and imaged with a multiphoton microscope. FIGS. 15B and 15D show the corresponding two-photon fluorescence images. Under two-photon pulse laser of 820 nm, bright green and red emission from cell cytoplasm could be clearly visualized, indicating that the internalized AIE-EGFR nanoparticles could be readily excited by two-photon laser, and provide excellent fluorescence for bioimaging. Considering excellent tissue penetration depth of two-photon fluorescence imaging, our AIE-EGFR nanoparticles could be used for detection integrin overexpressed tumors with improved in vivo resolution and detection sensitivity.

While this invention has been particularly shown and described with references to example embodiments thereof, it will be understood by those skilled in the art that various changes in form and details may be made therein without departing from the scope of the invention encompassed by the appended claims.

1. A nanoparticle composition comprising a plurality of surface conjugatable groups, wherein the nanoparticle comprises:
a) a biocompatible polymer shell having an average diameter of less than about 1000 nm;
b) a nanoparticle core encapsulated in the biocompatible polymer shell, the core comprising at least one uniform population of a photostable agent with aggregation-induced emission characteristic suitable for imaging applications;
c) at least one conjugatable group on the surface of the polymeric shell; and
d) at least one targeting moiety that can specifically bind to a target, the at least one targeting moiety covalently linked to the at least one conjugatable group.

2. The composition of claim 1, wherein the nanoparticle has an average diameter of about 50 nm to about 300 nm, about 20 nm to about 50 nm or about 10 nm to about 20 nm.

3.-4. (canceled)

5. The composition of claim 1, wherein the photostable agent with aggregation-induced emission characteristic has tunable absorption or emission wavelengths.

6. The composition of claim 1, wherein the photostable agent with aggregation-induced emission characteristic has a chemical structure set forth in any one of the formulae I-III: 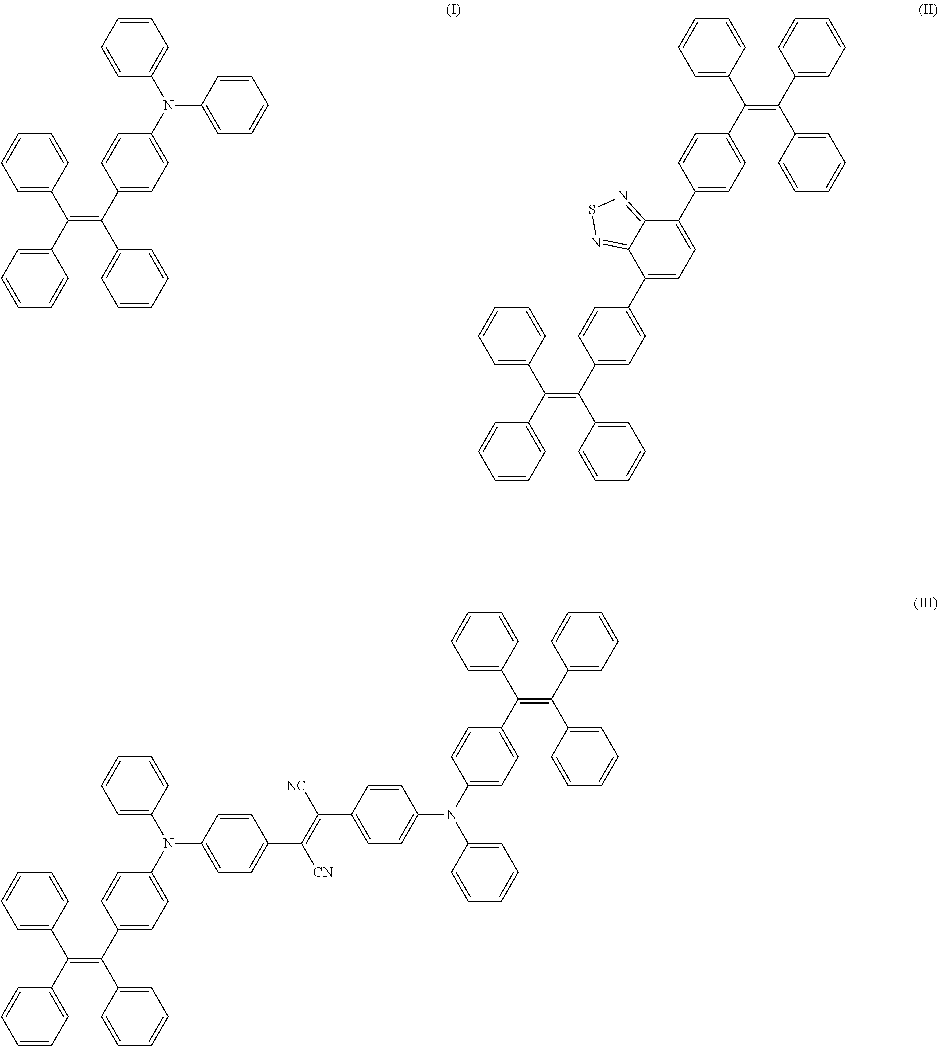 [SMILES: c1ccc(C(=C(c2ccccc2)c2ccc(N(c3ccccc3)c3ccccc3)cc2)c2ccccc2)cc1 ; c1ccc(C(=C(c2ccccc2)c2ccc(-c3ccc(-c4ccc(C(=C(c5ccccc5)c5ccccc5)c5ccccc5)cc4)c4nsnc34)cc2)c2ccccc2)cc1 ; N#C/C(=C(/C#N)c1ccc(N(c2ccccc2)c2ccc(C(=C(c3ccccc3)c3ccccc3)c3ccccc3)cc2)cc1)c1ccc(N(c2ccccc2)c2ccc(C(=C(c3ccccc3)c3ccccc3)c3ccccc3)cc2)cc1]
wherein at least one hydrogen atom on at least one of the tetraphenylethylene moieties is substituted with an electron group, such as methoxy, or an electron-withdrawing group, such as nitro or cyano.

7. The composition of claim 1, wherein the conjugatable group is an amine group, a carboxylic acid group, a sulfhydryl group, a maleimide group, an oxime group, alkyne, azide or combinations thereof.

8. The composition of claim 1, wherein the covalent linkage is a peptide linkage, an amide linkage, a sulfhydryl linkage, a maleimide linkage, a thioester linkage, an ether linkage, an ester linkage, a hydrazine linkage, a hydrazine linkage, an oxime linkage or combinations thereof.

9. The composition of claim 1, wherein the targeting moiety is a ligand, biomolecule, protein, a specific recognition element, a peptide, aptamer, antibody, affibody, antigen or antigen binding fragment thereof.

10. (canceled)

11. The composition of claim 9, wherein the antibody is an anti-EGFR antibody that binds to the epidermal growth factor receptor.

12. The composition of claim 9, wherein the affibody is an anti-her2 affibody.

13. The composition of claim 1, wherein the target is a surface antigen, ligand or receptor of a live cell.

14. A method for immunostaining or imaging a live cell, the method comprising:
a) contacting a live cell with a nanoparticle-target moiety complex, wherein the nanoparticle-target moiety complex comprises: a nanoparticle of claim 1 covalently linked to a targeting moiety is selected from a ligand, biomolecule, protein, a specific recognition element, a peptide, aptamer, antibody, affibody, antigen or antigen binding fragment thereof;
b) stabilizing the nanoparticle-target moiety complex that is bound to the live cell;
c) exciting the photostable agent in the nanoparticle-target moiety complex that is bound to the live cell with a laser source capable of producing light with a specific wavelength and collecting the images; and
d) processing the images, thereby imaging a live cell.

15. The method of claim 14, wherein the photostable agent with aggregation-induced emission characteristic has tunable absorption or emission wavelengths.

16. The method of claim 14, wherein the photostable agent with aggregation-induced emission characteristic has a chemical structure set forth in any one of the formulae I-III: 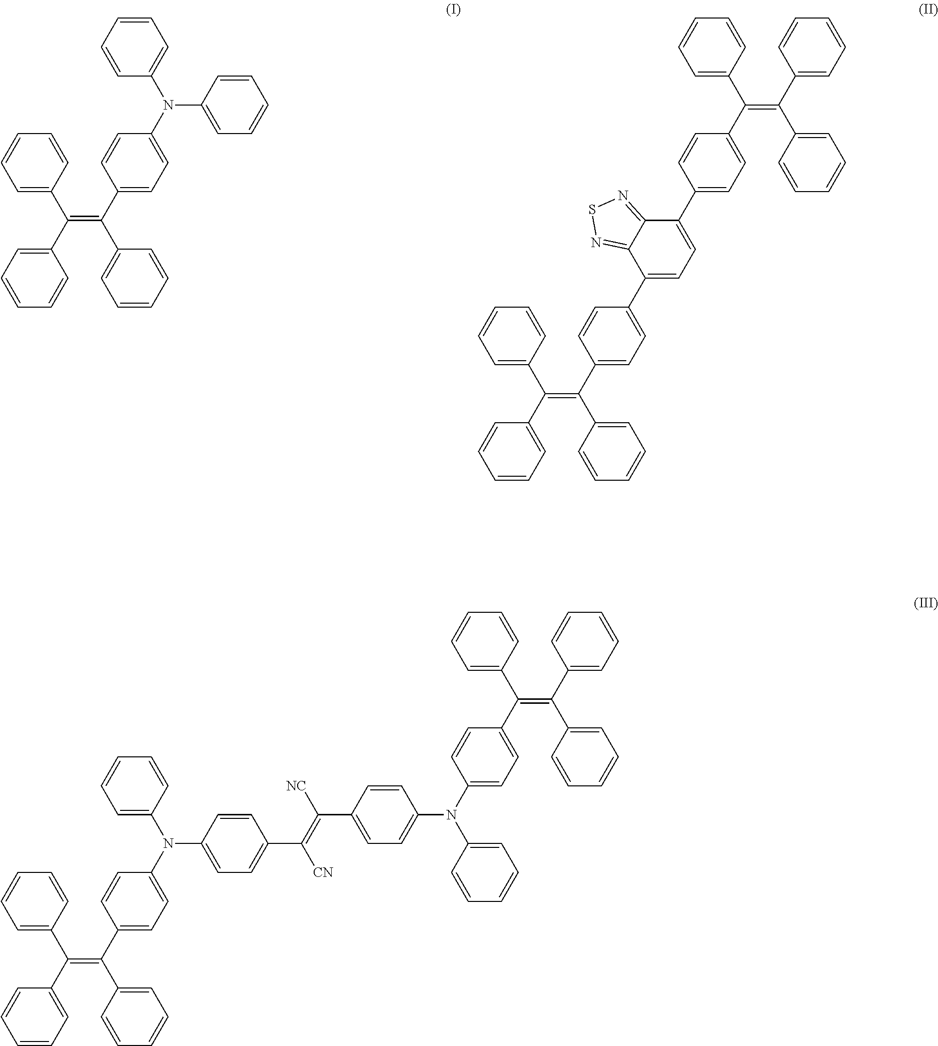 [SMILES: c1ccc(C(=C(c2ccccc2)c2ccc(N(c3ccccc3)c3ccccc3)cc2)c2ccccc2)cc1 ; c1ccc(C(=C(c2ccccc2)c2ccc(-c3ccc(-c4ccc(C(=C(c5ccccc5)c5ccccc5)c5ccccc5)cc4)c4nsnc34)cc2)c2ccccc2)cc1 ; N#C/C(=C(/C#N)c1ccc(N(c2ccccc2)c2ccc(C(=C(c3ccccc3)c3ccccc3)c3ccccc3)cc2)cc1)c1ccc(N(c2ccccc2)c2ccc(C(=C(c3ccccc3)c3ccccc3)c3ccccc3)cc2)cc1]
wherein at least one hydrogen atom on at least one of the tetraphenylethylene moieties is substituted with an electron group, such as methoxy, or an electron-withdrawing group, such as nitro or cyano.

17.-18. (canceled)

19. The method of claim 14, wherein the antibody is an anti-EGFR antibody that binds to the epidermal growth factor receptor.

20. The method of claim 14, wherein the affibody is an anti-her2 affibody.

21. (canceled)

22. A method for controlling the size of a nanoparticle, comprising:
a) varying the loading ratio of the polymer to the dyes with aggregation induced emission;
b) changing the solvents and solvent ratio used for the formulation of the nanoparticles; and
c) changing the ratio of the hydrophilic to hydrophobic length of the polymer, to thereby control the size of a nanoparticle.

23. AIE nanoparticle comprising a DSPE core and PEG shell conjugated to a targeting moiety, the AIE nanoparticle-targeting moiety is selected from the following: AIE nanoparticle-antibody, AIE nanoparticle-affibody, AIE nanoparticle-protein, AIE nanoparticle-peptide, AIE nanoparticle-aptamer, AIE nanoparticle-antigen, or AIE nanoparticle-antigen binding fragment or AIE nanoparticle-target ligands; wherein the AIE fluorogen is one of the following:

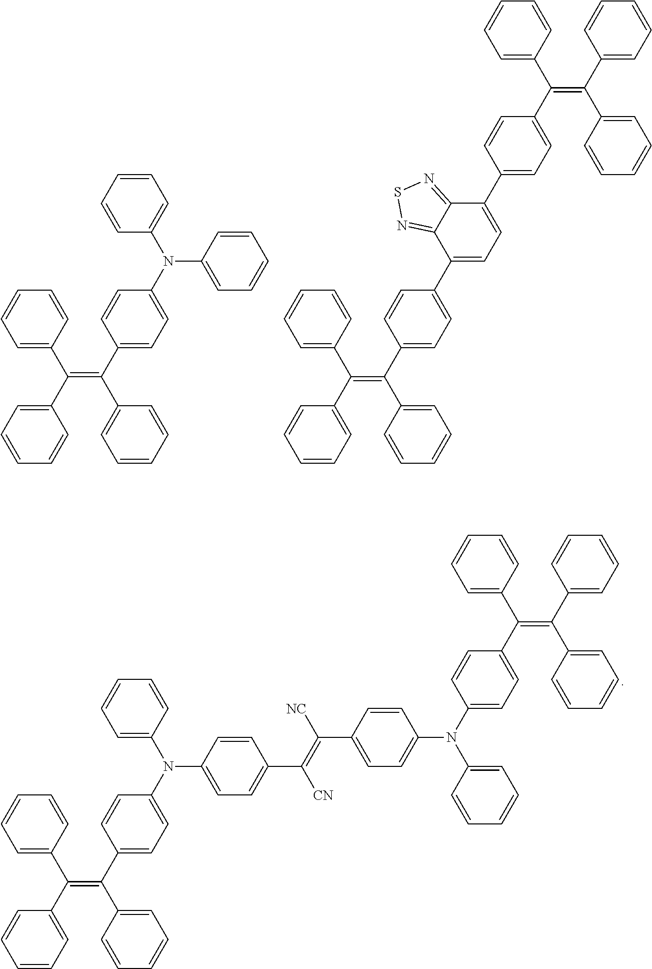

24. A method for designing an AIE nanoparticle, comprising:
selecting an AIE fluorogen that fluoresces at a desired wavelength;
selecting a conjugatable group and linker that can be covalently linked to at least one targeting moiety; and
controlling the size of the nanoparticle using the method of claim 22.

25.-27. (canceled)

28. Kit for AIE nanoparticle conjugation to a targeting moiety, comprising:
a) surface functionalized AIE nanoparticle of claim 1 where the targeting moiety is not present;
b) conjugation buffer,
c) washing buffer; and
d) instructions for performing the conjugation reaction.

29. A method of immunostaining comprising using the AIE conjugate of claim 23, wherein the conjugate is AIE-IgG.

30. A method for cancer cell detection comprising using the AIE conjugate of claim 23, wherein the conjugate is AIE-HER2 and/or AIE-EGFR.

31. A method for cancer cell tracking comprising using the AIE conjugate of claim 23, wherein the conjugate is AIE-HER2.
